(19) United States
(12) Patent Application Publication
(10) Pub. No.: US 2011/0006904 A1
(43) Pub. Date: Jan. 13, 2011

(54) MANAGING A SET OF SWIMMING POOLS

(75) Inventors: Lionel DUCHAMP, Gruffy (FR); Omar Saaid, Maison Laffite (FR); Jean-Marc Boutet, Le Pecq (FR)

Correspondence Address: FROMMER LAWRENCE & HAUG, 745 FIFTH AVENUE- 10TH FL., NEW YORK, NY, 10151, US

(73) Assignee: Klereo, Montesson (FR)

(21) Appl. No.: 12/833,614

(22) Filed: Jul. 9, 2010

(30) Foreign Application Priority Data
Jul. 10, 2009 (FR) 09/54840

(51) Int. Cl. G08B 21/18; G05B 23/02; G01N 33/18

(52) U.S. Cl. 340/603; 340/3.1; 73/53.01

(57) ABSTRACT

Method for managing a set of swimming pools, each swimming pool being provided with a means of monitoring the quality of the water contained in the swimming pool, each of the monitoring means communicating by means of a communication network with at least one management centre, characterized in that it comprises a first step of detection of a degraded state on at least one swimming pool of the set, called “victim swimming pool”, and a second step of determining swimming pools of the set threatened with turning into a degraded state, each called “threatened swimming pool”. Management centre for a set of swimming pools implementing such a method, device for managing a subset of a set of swimming pools, means of monitoring the quality of the water contained in a swimming pool and use of a swimming pool as sensor to determine a potential risk.

This application claims priority to French Patent Application Number 09 54840 filed Jul. 10, 2009, the disclosure of which is incorporated herein by reference.

The invention relates to the field of remote management of a set of individual swimming pools. An individual swimming pool is provided with means of monitoring the quality of the water contained in the swimming pool. Such monitoring means comprises physico-chemical or bacteriological means of analysing the water and ambient parameters and treatment means such as dosing pump, ozone generator, ultraviolet lighting, circulation motor, filters, etc.

The monitoring means may be sufficiently well designed to ensure good quality water without the intervention of the owner of the swimming pool or at least with limited interventions and/or guiding him in said interventions to simplify them as much as possible.

However, the servicing of such a swimming pool is often entrusted to a service agent charged with periodically monitoring the correct operation of the monitoring means and the quality of the water, notably during periods of absence of the owner if it is a swimming pool installed in a second home.

One problem associated with the quality of the water of a swimming pool is the possibility of the water turning into a modified state in which the water becomes cloudy (the water is said to have “turned”), this state being qualified here as degraded state. Such a degraded state requires a complete draining and refilling of the swimming pool basin or requires a curative treatment that is intense, costly and possibly poisonous for the users or for the environment because of the quantity of chemical products to be used.

There are very many physico-chemical or bacteriological factors that can cause the appearance of a degraded state, and several types of such states may occur, which makes preventing the appearance of a degraded state very complex.

The inventors have nevertheless observed that there is a proximity relationship between a swimming pool that has just entered into a degraded state and swimming pools threatened with shifting very rapidly themselves into the same state.

Thus, according to the invention, it becomes possible for a management centre for a set of individual swimming pools to use as “sensor” a swimming pool that has undergone a change of state, called “victim swimming pool”, and determine the swimming pools that are threatened themselves with turning into a degraded state.

According to the invention, the method for managing a set of swimming pools, each swimming pool being provided with a means of monitoring the quality of the water contained in the swimming pool, each of the monitoring means communicating by means of a communication network with at least one management centre, comprises a first step of detection of a degraded state on at least one swimming pool of the set, called “victim swimming pool”, and a second step of determining swimming pools of the set threatened with turning into a degraded state, each called “threatened swimming pool”.

According to the invention, the management centre can communicate by means of the communication network with at least one device for managing at least one swimming pool and the management method may comprise an alert third step, in which the management centre sends a first alert message to the managing device, the first alert message indicating the identity of at least one threatened swimming pool.

According to the invention, in the third step, the management centre can send as many first customized alert messages as there are managing devices, each managing device being associated with a subset of the set of swimming pools, the first alert message being sent to a managing device only if at least one swimming pool of the subset is a threatened swimming pool.

According to the invention, the management method may comprise a signalling fourth step, in which the management centre sends a second alert message to the monitoring means of a threatened swimming pool, this monitoring means comprising at least one signalling means capable of transmitting an alert signal to a user, the second message containing threat type information and/or containing a preventive treatment indication.

According to the invention, the management method may comprise a direct activation fifth step in which the monitoring means of a threatened swimming pool receives a preventive treatment command via the network.

According to the invention, in the second step of the management method, the determination of threatened swimming pools may take into account the location of a swimming pool, called “relative swimming pool”, relative to the victim swimming pool to determine whether the relative swimming pool is a threatened swimming pool.

According to the invention, the determination of threatened swimming pools may use a risk criterion that decreases with the value of the distance between the relative swimming pool and the victim swimming pool and/or that decreases with the value of the measured angle between a first direction passing through the victim swimming pool and oriented in the wind direction and a second direction passing through the victim swimming pool and the relative swimming pool.

According to the invention, the management centre for a set of swimming pools equipped with means of communicating via a communication network with means of monitoring the quality of the water of the swimming pools and with at least one device for managing a number of swimming pools, comprises hardware and software means capable of implementing the management method as described hereinabove.

According to the invention, a device for managing a subset of a set of swimming pools, connected to a communication network, comprises a human-machine interface and hardware and software means capable of:
storing an identifier of each swimming pool belonging to the subset,
communicating over the communication network with a means of monitoring the quality of the water of each swimming pool belonging to the subset,
communicating over the communication network with a management centre for a set of swimming pools, known to the managing device, and applying the management method as described hereinabove,
   and, following the reception of an alert message originating from the management centre and containing an identifier of a swimming pool threatened with turning into a degraded state, capable of executing at least one of the following operations if the identifier is one of the stored identifiers:
using the human-machine interface to display the alert message and/or to display information related to the threatened swimming pool identifier,
using the communication network to send to the monitoring means of the threatened swimming pool a preventive treatment command and/or a signalling command and/or all or part of the alert message.

According to the invention, a means of monitoring the quality of the water contained in a swimming pool, connected to a communication network, comprises signalling means and hardware and software means capable of:
saving an identifier of the swimming pool in a memory of the monitoring means,
communicating over the communication network with a management centre for a set of swimming pools, known to the monitoring means, and applying the management method as described hereinabove,
communicating over the communication network with a device for managing a subset of the set of swimming pools, known to the monitoring means,
   and capable of executing at least one of the following two operations:
following the reception of an alert message, originating from the management centre or originating from the managing device and containing the identifier, using the signalling means to signal a risk of turning into a degraded state and/or a preventive treatment indication,
following the reception of a preventive treatment command from the management centre or from the managing device, executing the preventive treatment command.

According to the invention, a swimming pool of a set of swimming pools is used as sensor to determine the potential risk on other swimming pools of the set when the swimming pool turns into a degraded state.

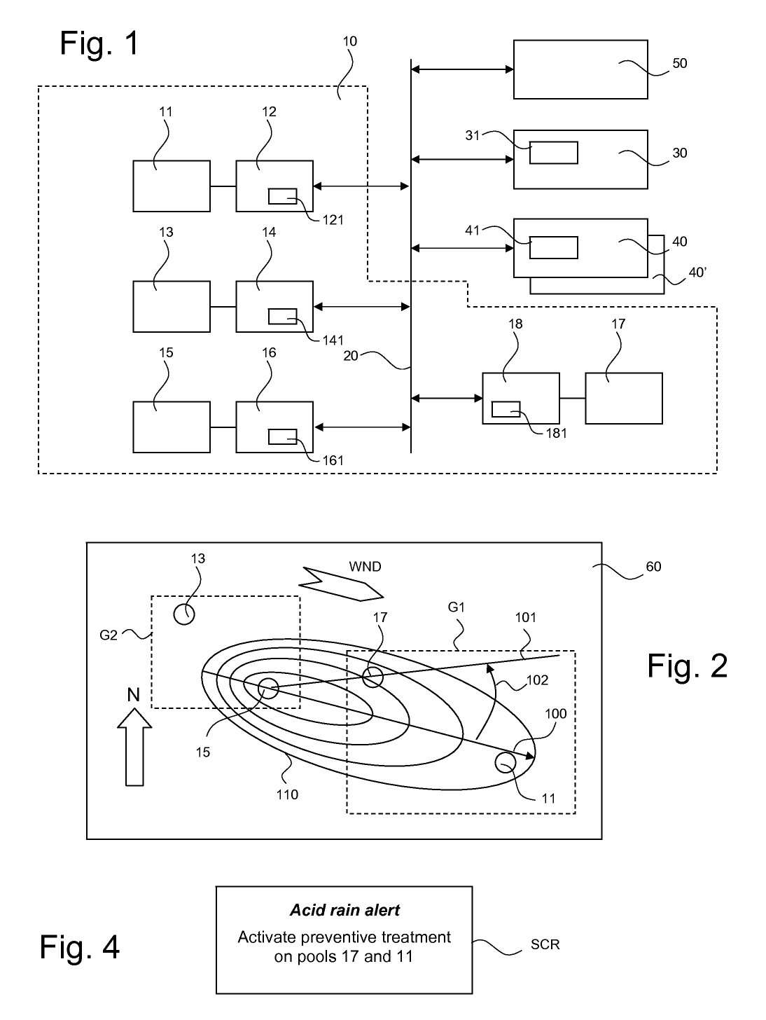

FIG. 1 represents a set of swimming pools and its link via a communication network with a management centre for this set of swimming pools, managing devices and an information base.

FIG. 2 represents a geographic map indicating the position of a victim swimming pool and of threatened swimming pools.

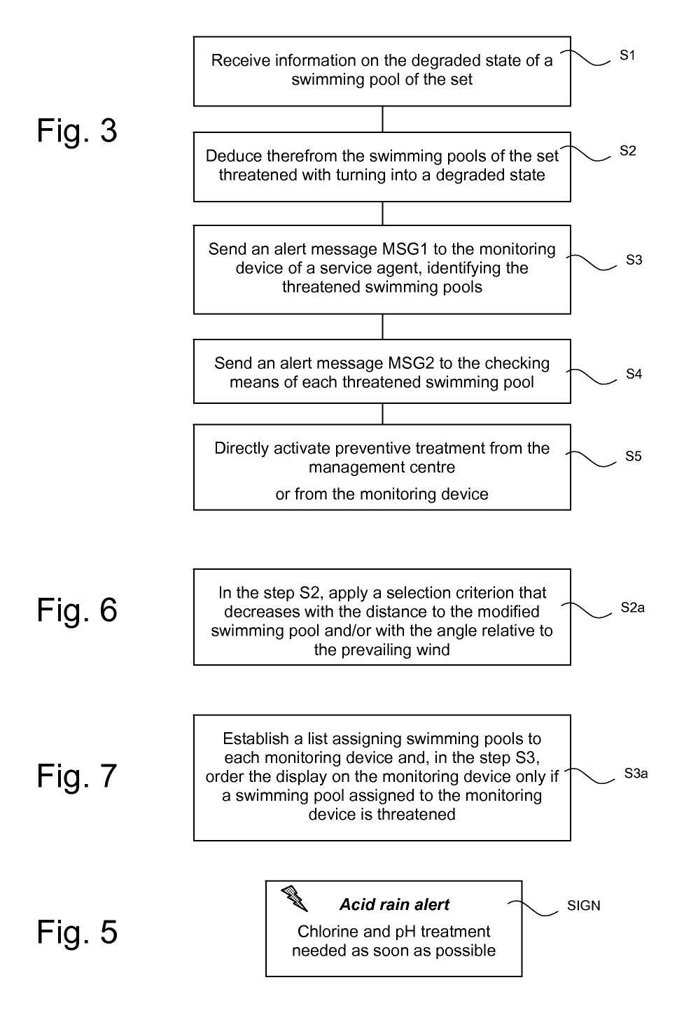

FIG. 3 represents a method of managing a set of swimming pools, according to the invention.

FIG. 4 represents the display of a first alert message on a managing device.

FIG. 5 represents the display of a second alert message, on a monitoring means.

FIG. 6 represents a complementary variant of a second step of the management method.

FIG. 7 describes a complementary variant of a third step of the management method.

FIG. 1 represents a set of swimming pools 10 and its link via a communication network 20 with a management centre 30 for this set of swimming pools, managing devices 40 and 40′ and an information base 50.

The set of swimming pools comprises four swimming pools, of uncovered individual swimming pool type, installed in a main or second home. Each swimming pool comprises a means of monitoring the quality of the water as described in the prior art, provided with analysis means and treatment means that are not represented.

Thus, a first swimming pool 11 is connected to a first monitoring means 12, a second swimming pool 13 is connected to a second monitoring means 14, a third swimming pool 15 is connected to a third monitoring means 16, a fourth swimming pool 17 is connected to a fourth monitoring means 18.

Each monitoring means comprises signalling means, respectively 121, 141, 161, 181, for the monitoring means 12 to 18. For simplicity, each monitoring means is represented in the form of a single block but it should be understood that it comprises a number of subsystems, possibly dissociated and likely to intercommunicate by wired, infrared or microwave channel.

The swimming pools and the monitoring means may be structurally different from one swimming pool to another. Thus, the set of swimming pools constitutes a set of heterogeneous items. However, all the monitoring means are linked to one and the same network 20, for example, and preferably, a public network of internet type. A management centre 30 for the set of swimming pools is also linked to this network.

The definition of the set results in fact from individual subscriptions to the management centre and is reflected at least in the presence of identifiers of each monitoring means in a memory 31 of the management centre. Conversely, an identifier of the management centre is known to each monitoring means.

Thus, other swimming pools, possibly geographically close to the swimming pools 11, 13, 15 and 17, do not belong to the set because of the absence of subscription to the management centre, and there is no reciprocal knowledge of identifiers between the monitoring means of one of these other swimming pools and the management centre.

In addition to an identifier of each monitoring means of swimming pools of the set, the management centre holds in memory precise data concerning the location of the swimming pool, its dimensional characteristics, the characteristics of the monitoring means and, possibly, it also has history data concerning the physico-chemical or bacteriological state of the water and/or concerning maintenance operations.

A first managing device 40 is also connected to the network. This managing device is used by a first swimming pool maintenance professional, called “service agent” or even “swimming pool expert”, who has signed a service agreement with some of the owners of swimming pools of the set, for example those of the first swimming pool 11 and of the fourth swimming pool 17, defining a subset G1 within the set.

In the same way, a second managing device 40′ is used by a second swimming pool maintenance professional who has signed a service agreement with the owners of the second swimming pool 13 and of the third swimming pool 15, defining a subset G2 within the set.

Each managing device is known both to the management centre and to the monitoring means of the concerned subset, at least by a sharing of identifiers.

A managing device comprises a human-machine interface and holds in memory, like the management centre, precise data concerning the location of the swimming pool, its dimensional characteristics, the characteristics of the monitoring means and, possibly, it also has history data concerning the physico-chemical or bacteriological state of the water and/or concerning maintenance operations. These data are preferably saved by the professional in his managing device and then communicated to the management centre.

Because of the possibility of bidirectional communication over the network and of the presence of suitable hardware and software means in the monitoring means, in the management centre and in the managing devices, it is possible, from the management centre or from a managing device, to have access to the data measured by a monitoring means and/or initiate remote treatment operations on this monitoring means. A monitoring means may also directly warn the corresponding managing device and the management centre in the event of a serious event, and notably in the case of a turn into a degraded state.

An information base 50 is also linked to the network. This may be a weather server.

FIG. 2 represents a geographic map 60 indicating the position of a victim swimming pool and of threatened swimming pools. The map is oriented in the direction of geographic north N. It represents all the swimming pools of the set. For simplicity and in order to illustrate the principle of the invention, the set comprises only four swimming pools, but the invention is all the more relevant with a large number of swimming pools belonging to the set. In this case, the map may also represent only a portion of the set, in zoom form.

The monitoring means 16 of the third swimming pool 15 has detected the appearance of a degraded state and immediately transmits the information to the management centre. The swimming pool 15 is therefore victim. There is a strong probability that this change of state is the consequence of pollution by acid rain or another local weather phenomenon, likely to contaminate in the same way any swimming pool placed in the same conditions. The swimming pool 15 therefore constitutes a “sensor” locating a focus of risk.

The assessment of the risk on the swimming pools of the set uses a risk criterion. This risk criterion is at least one that decreases with the distance between a relative swimming pool, for example the fourth swimming pool 17, and the victim swimming pool. Thus, a simple first criterion varies for example from 1 (for a distance less than 10 km) to 0 (for a distance greater than 100 km).

However, it is useful to take into consideration the direction of the wind in the risk criterion. On the map 60, the direction of the wind is shown in the form of a solid arrow WND. This information is obtained by the management centre from the information base 50.

A first direction 100 is defined that passes through the victim swimming pool and is oriented in the direction of the wind and a second direction 101 that passes through the victim swimming pool and the relative swimming pool and is oriented in this direction. An angle 102 measured between these two directions varies between 0° when the relative swimming pool is downwind of the victim swimming pool and 180° when the relative swimming pool is upwind of the victim swimming pool. The risk criterion decreases when the angle 102 increases. Thus, a simple second criterion varies for example from 1 to 0 when the angle varies from 0° to 180°.

Preferentially, the risk criterion takes into account both the distance and the direction relative to the wind, for example by calculating the product of the two simple criteria.

On the map 60, several of the same risk (iso-risk) curves are represented, such as, for example, the iso-risk curve 110, in the form of ellipses of which the victim swimming pool is a focus and the long axis is oriented in the direction of the wind. It is assumed that the relative swimming pool 17 is such that the risk criterion exceeds a predetermined threshold. The third swimming pool 17 is therefore threatened. Similarly, it is assumed that the first swimming pool 11 is also threatened. However, the second swimming pool 13, although situated roughly at the same distance from the victim swimming pool as the third swimming pool 17, is not threatened, the risk criterion for it being below the predetermined threshold.

Given a stronger wind, the eccentricity of the ellipses increases, and the risk becomes zero in the upwind direction if the wind is the sole cause of the change of state.

FIG. 3 represents a management method for a set of swimming pools, according to the invention.

In a first step S1, the management centre acquires information that a swimming pool has turned into a degraded state. This information is, for example, transmitted directly to the management centre by the monitoring means of the swimming pool that has undergone this change of state. Alternatively, the information is automatically transmitted by the monitoring means to the managing device which relays the information to the management centre.

In a second step S2, the software means of the management centre then apply a programme for determining threatened swimming pools. This programme uses a risk criterion as explained previously and represented in FIG. 6. The programme may also include recourse to an expert system, also incorporating relief data and/or history data. Some swimming pools may thus be known to exhibit a stronger sensitivity because of a particular constitution or because of the monitoring means used or even without there being any particular explanation but simply because of historical incidents. The predetermined threshold may therefore be adjusted according to this sensitivity specific to each swimming pool and acquired, for example automatically, using the expert system.

The risk criterion may also be increased by the simultaneous presence of two, or even more, victim swimming pools in one and the same area. In a variant of the method, it is considered that there is a threat only if at least two swimming pools of one and the same area are victims and if one and the same type of turn into a degraded state has been detected for these two swimming pools. A list of threatened swimming pools is established.

In a third step S3, the management centre sends a first alert message MSG1 to the professional's managing device. This first alert message indicates the identity of the threatened swimming pools, for example the swimming pools 11 and 17 if referring to the case of FIG. 2. Preferentially, the type of threat is indicated in the first alert message, as is the type of preventive treatment to be applied. The first alert message may contain this information in coded form, but the managing device may convert this coded message into an easily readable alert screen or even an audible message. FIG. 4 represents by way of example the display of an alert screen SCR on the human-machine interface 41 of the first managing device 4 following the reception of the first alert message.

In a fourth step S4, the management centre sends a second alert message MSG2 to the monitoring means of the threatened swimming pools, namely, respectively, the monitoring means 12 and 18. As previously, the second message may be coded, but it results in the transmission of a comprehensible signal to a user, for example the owner of the threatened swimming pool. FIG. 5 represents, by way of example, the display of an alert signal SIGN on the signalling means of the monitoring means of the first swimming pool 11. The alert message preferentially contains information concerning the nature of the treatment to be applied.

To avoid unnecessarily bothering the owner of the swimming pool, the fourth step is either optional or implemented only if the management centre observes the absence of reaction on the part of the managing device, or even implemented with a certain delay, for example a few hours after the third step.

It is also possible to transmit a message directly to the owner by SMS-type digital telephone messaging.

In a fifth step S5, also optional, the appropriate preventive treatment is implemented directly by the management centre. The preventive treatment may also be implemented remotely by the professional using his managing device. In both cases, the monitoring means of the threatened swimming pool receives a preventive treatment command via the network.

An option of the monitoring means enables the owner to accept or deny direct activation of the monitoring means by the management centre or the managing devices.

It is important to note that a preventive treatment requires much less in the way of chemical products and/or energy than a curative treatment.

A preventive treatment may also involve the closure of a moving screen protecting the uncovered swimming pool.

As seen above, FIG. 4 represents the display of an alert screen SCR on a managing device and FIG. 5 represents an alert signal SIGN, sent by a signalling means of a monitoring means. In both cases, an LCD or OLED type display may be used.

These messages may be sent by the management centre in the form of alphanumeric strings. Preferentially, at least some of the message is saved in a memory of the managing device or of the monitoring means. For example, the content of the signal SIGN is fully pre-saved in the monitoring means 12 as is, for example, the preventive treatment algorithm. The owner simply confirms the starting up of the preventive treatment by pressing a key. In the second alert message MSG2, the management centre then simply sends a code resulting in particular in the display of the alert signal. An example of an alert signal, partially pre-saved, is as follows:

### “Alert” [threat_type]

### “—treatment” [treatment_type] “necessary as soon as possible. Press confirmation key”

In this example, the management centre simply sends to the monitoring means the coded content of the variables [threat_type] and [treatment_type]. The monitoring means holds in memory the various alphanumeric strings corresponding to the various codes and shows the message corresponding to the code received.

Examples of alphanumeric values for [threat_type]=“acid rain” or “sandstorm” or “pollution”, etc.

Examples of alphanumeric values for [treatment type]=“preventive” or “chlorine” or “chlorine and pH” or “UV”, etc.

FIG. 6 represents a complementary variant S2a of the second step S2 of the management method, described previously in relation to this second step.

FIG. 7 describes a complementary variant S3a of the third step S3 of the management method.

The hardware and software means of the management centre are used to apply the management method and can also be used to store a mapping table between identifiers of each monitoring means and each managing device, that is to say a relationship assigning each swimming pool of the set to each professional. When applying the second step, the management centre orders the display on a managing device only if a swimming pool assigned to the managing device is threatened. For example, the first message MSG1 will be sent only to the first managing device 41 because it concerns only the subset G1 managed by this device. No message is sent to the second managing device.

If the first swimming pool 11 belonged to the second subset G2, then only the number 17 would appear in the screen SCR of FIG. 4.

The management method is therefore open to numerous variants, notably by the fact that the messages and/or the commands are directly transmitted by the management centre to the monitoring means or are transmitted to the managing device which relays them or not to the monitoring means, wholly or partly or after reprocessing.

According to the invention, the management centre is the system in which the identity of the threatened swimming pools is determined, once the identity of a victim swimming pool is known.

In a set of swimming pools comprising relatively few individual swimming pools, a single installer manages the set of swimming pools. His managing device however becomes “management centre” within the meaning of the invention, if said device contains an algorithm capable of determining the risks to swimming pools of the set based on the reception of information that one of the swimming pools of the set has turned into a degraded state.

The invention is independent of the nature of the communication network. The latter may be homogeneous or heterogeneous. For example, in the first case, all the communications are handled via the internet or all the communications are handled by the sending of SMSs, whereas in the second case some communications may be handled via the internet whereas other communications may be handled by the sending of SMSs.

1. Method for managing a set of swimming pools, each swimming pool being provided with a means of monitoring the quality of the water contained in the swimming pool, each of the monitoring means communicating by means of a communication network with at least one management centre of the set of swimming pools, wherein it comprises a first step (S1) of detection of a degraded state on at least one swimming pool of the set, called “victim swimming pool”, and a second step (S2) of determining swimming pools of the set threatened with turning into a degraded state, each called “threatened swimming pool”.

2. Management method according to claim 1, wherein the management centre communicates by means of the communication network with at least one device for managing at least one swimming pool and in that it comprises an alert third step (S3), in which the management centre sends a first alert message to the managing device, the first alert message indicating the identity of at least one threatened swimming pool.

3. Management method according to claim 2, wherein, in the third step (S3a), the management centre sends as many first customized alert messages as there are managing devices, each managing device being associated with a subset of the set of swimming pools, the first alert message being sent to a managing device only if at least one swimming pool of the subset is a threatened swimming pool.

4. Management method according to claim 1, wherein it comprises a signalling fourth step (S4), in which the management centre sends a second alert message to the monitoring means of a threatened swimming pool, this monitoring means comprising at least one signalling means capable of transmitting an alert signal to a user, the second message containing threat type information and/or containing a preventive treatment indication.

5. Management method according to claim 1, wherein it comprises a direct activation fifth step (S5) in which the monitoring means of a threatened swimming pool receives a preventive treatment command via the network.

6. Management method according to claim 1, wherein, in the second step, the determination of threatened swimming pools takes into account the relative location of a swimming pool, called “relative swimming pool”, relative to the victim swimming pool to determine whether the relative swimming pool is a threatened swimming pool.

7. Management method according to claim 6, wherein the determination of threatened swimming pools uses a risk criterion that decreases with the value of the distance between the relative swimming pool and the victim swimming pool and/or that decreases with the value of the measured angle between a first direction passing through the victim swimming pool and oriented in the wind direction and a second direction passing through the victim swimming pool and the relative swimming pool.

8. Management centre for a set of swimming pools equipped with means of communicating via a communication network with means of monitoring the quality of the water of the swimming pools and with at least one device for managing a number of swimming pools, wherein it comprises hardware and software means capable of implementing the management method according to claim 1.

9. Device for managing a subset of a set of swimming pools, connected to a communication network, wherein it comprises a human-machine interface and hardware and software means capable of:
storing an identifier of each swimming pool belonging to the subset,
communicating over the communication network with a means of monitoring the quality of the water of each swimming pool belonging to the subset,
communicating over the communication network with a management centre for a set of swimming pools, known to the managing device, and applying the management method of claim 1,
and, following the reception of an alert message originating from the management centre and containing an identifier of a swimming pool threatened with turning into a degraded state, capable of executing at least one of the following operations if the identifier is one of the stored identifiers:
using the human-machine interface to display the alert message and/or to display information related to the threatened swimming pool identifier,
using the communication network to send to the monitoring means of the threatened swimming pool a preventive treatment command and/or a signalling command and/or all or part of the alert message.

10. Means of monitoring the quality of the water contained in a swimming pool, connected to a communication network, wherein it comprises signalling means and hardware and software means capable of:
saving an identifier of the swimming pool in a memory of the monitoring means,
communicating over the communication network with a management centre for a set of swimming pools, known to the monitoring means, and applying the management method of claim 1,
communicating over the communication network with a device for managing a subset of the set of swimming pools, known to the monitoring means;
and capable of executing at least one of the following two operations:
following the reception of an alert message, originating from the management centre or originating from the managing device and containing the identifier, using the signalling means to signal a risk of turning into a degraded state and/or a preventive treatment indication,
following the reception of a preventive treatment command from the management centre or from the managing device, executing the preventive treatment command.
